(19) United States
(12) Patent Application Publication
(10) Pub. No.: US 2009/0047223 A1
(43) Pub. Date: Feb. 19, 2009

(54) Improvements in or Related to Organic Compoounds

(75) Inventors: Andreas Natsch, Uetikon (CH); Felix Flachsmann, Duebendorf (CH)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 12/097,896

(22) PCT Filed: Dec. 14, 2006
(86) PCT No.: PCT/CH2006/000700

§ 371 Date: Jun. 18, 2008

(30) Foreign Application Priority Data
Dec. 23, 2005 (GB) 0526279.5

(51) Int. Cl. A61K 8/365; C07C 61/08; A61P 1/02

(52) U.S. Cl. 424/55; 562/509

(57) ABSTRACT

Malodour counteracting preparations for oral use comprising esterified fumarates of the formula (I) wherein X and Y have the same meaning as given in the description, is disclosed. Furthermore, the invention refers to a process for their preparation and to their use for preventing or reducing oral malodour.

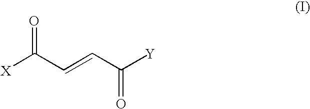

The present invention refers to malodour counteracting preparations for oral use comprising esterified fumarates, to processes for their preparation and to their use for preventing or reducing oral malodour.

Oral malodour is formed by microorganisms in the oral cavity. Main components causing halitosis comprise volatile sulphur compounds (VSCs) including, for example, hydrogen sulphide (H₂S), methanethiol (CH₃SH), dimethyl mercaptan ((CH₃)₂S) and the like. Particularly, methyl mercaptan is known as a main compound of offensive odor contributing to halitosis due to its very low odor threshold value, which is defined as the lowest concentration of the vapor of an odorous material in the air which can be detected. Sulfide compounds, which are contained in hot pepper or ingested garlic, such as allyl mercaptan, are also responsible for oral malodour.

Several possibilities for combatting oral malodour have been described in literature. One possibility is the use of oral products comprising intense flavours to mask oral malodour. Another option is the use of oral care products comprising antibacterial agents, both natural ingredients such as mint oils, thymol, eucalyptol and eugenol, and artificial compounds such as chlorhexidine, either alone or combinations thereof. A further way to combat halitosis is by enzymatic inhibition of the relevant bacterial enzyme(s), so that the volatile sulphur compounds are not formed in the first place.

A further alternative for combatting oral malodour is the use of compounds that have the ability to capture volatile sulphur compounds. Examples include zinc salts and polyphenols, of the type found in green tea. The capability of fumaric acid esters to bind malodorous substances present in the ambient air by chemical reaction has been known for a long time. For example, U.S. Pat. No. 3,077,457 describes the deodorization of a space by spraying into the space a composition comprising a di-ester of fumaric acid, such as dibutyl fumarate, dihexyl fumarate, digeranyl fumarate or dibenzyl fumarate. These compositions have been found to reduce tobacco smoke odor and kitchen odor. The use of C₁₋₃ dialkyl fumarate and C₂₋₃ dialkenyl fumarate for deodorising air is described in GB 1401550. The use of certain aromatic unsaturated carboxylic acid esters in combination with alkyl fumarates as malodor counteractants is disclosed in WO02/051788.

The methods known in the art for combatting oral malodor are only partially successful and there still remains a need for further options which are even more efficient against oral malodor.

Surprisingly, the inventors now found a new class of compounds capable of neutralising oral malodor combining two different mechanisms. On the one hand the compounds of the present invention are capable of chemically binding the volatile sulphur compounds and on the other hand the compounds have the capability of releasing an organoleptic compound in small amounts over a long time period. The released organoleptic compound in turn may mask oral malodor. Extensive studies revealed that, among fumaric acid derivatives, only compounds which are sufficiently hydrophilic have the ability to be active in the oral cavity against oral malodor.

Thus the present invention refers in one of its aspects to oral compositions comprising a compound of formula (I)

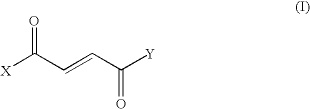

wherein
X is the residue of an organoleptic alcohol comprising 8 to 15 carbon atoms; or
X is the residue of an alcohol, diol, triol or polyol comprising 2 to 7 carbon atoms; and
Y is the residue of an organoleptic alcohol comprising 8 to 15 carbon atoms;
the compounds of formula (I) having a CLogP of 4.5 or lower; and
the double bond between the two carboxylic groups is preferably of E configuration.

The term “CLogP” is used herein for the calculated n-octanol/water partition coefficient, calculated using ChemDraw® Ultra 8.0 software from CambridgeSoft Corporation, Cambridge (USA) which is based on the CLogP algorithm from BioByte Corporation.

In a preferred embodiment, the invention refers to oral compositions comprising a compound of formula (I)

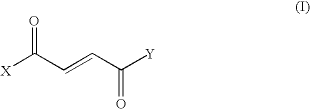

wherein
X is the residue R¹—O of an organoleptic alcohol of the formula R¹—OH, wherein R¹ is selected from the group consisting of
I) saturated and unsaturated, linear and branched, C₈-C₁₅ hydrocarbon residues, optionally containing one or more hydroxyl, carbonyl, carboxyl, and or ether group(s);
II) C₈-C₁₃ hydrocarbon residue containing one ring structure selected from alicyclic C₅, alicyclic C₆, phenol, bicyclic C₇, furan, and spirocyclic C₉ wherein one ring member is an oxygen,
and wherein the C₈-C₁₃ hydrocarbon residue optionally contains one or more hydroxyl, carbonyl, carboxyl, and or ether group(s); or
   X is the residue R²—O of ascorbic acid or an alkanol R²—OH, wherein R² is saturated or unsaturated, linear or branched C₂-C₇ alkyl optionally containing one or more hydroxyl, ether, and/or carbonyl group(s), or R² is a C₃-C₇ cycloalkyl optionally containing one or more hydroxyl and/or carbonyl group(s); and
   Y is the residue R³—O of an organoleptic alcohol of the formula R³—OH, wherein R³ is selected from the group consisting of
I) saturated and unsaturated, linear and branched, C₈-C₁₅ hydrocarbon residues, optionally containing one or more hydroxyl, carbonyl, carboxyl, and or ether group(s);
II) C₈-C₁₃ hydrocarbon residue containing one ring structure selected from alicyclic C₅, alicyclic C₆, phenol, bicyclic C₇, furan, and spirocyclic C₉ wherein one ring member is an oxygen,
and wherein the C₈-C₁₃ hydrocarbon residue optionally containing one or more hydroxyl, carbonyl, carboxyl, and or ether group(s);
   the compounds of formula (I) having a CLogP of 4.5 or lower; and the double bond between the two carboxylic groups is preferably of E configuration.

Examples of organoleptic alcohols R¹—OH/R³—OH from which the residues

Y and X respectively are derived are:
2-isopropyl-5-methylcyclohexanol; 2-isopropenyl-5-methyl-cyclohexan-2-ol; 2-isopropyl-5-methyl-phenol; 1,7,7-trimethyl-bicyclo[2.2.1]heptan-2-ol; 5-isopropyl-2-methyl-phenol; 2-isopropyl-5-methyl-phenol; 5-isopropenyl-2-methyl-cyclohex-2-enol; 1-isopropyl-4-methyl-cyclohex-3-enol; 2-hydroxy-succinic acid diethyl ester; 5-isopropenyl-2-methyl-cyclohexanol; 2-isopropenyl-5-methyl-cyclohexanol; 2-methyl-1-phenyl-propan-2-ol; 4-ethyl-2-methoxy-phenol; 4-allyl-2-methoxy-phenol; 3,7,11-trimethyl-dodeca-2,6,10-trien-1-ol; 1,3,3-trimethyl-bicyclo[2.2.1]heptan-2-ol; 3,7-dimethyl-octa-2,6-dien-1-ol; 4-(4-hydroxy-phenyl)-butan-2-one; (4-isopropenyl-cyclohex-1-enyl)-methanol; 2-phenyl-propan-1-ol; 3,7,11-trimethyl-dodeca-1,6,10-trien-3-ol; (4-isopropyl-phenyl)-methanol; 4-(4-hydroxy-3-methoxy-phenyl)-butan-2-one; 6-isopropyl-3-methyl-cyclohex-2-enol; 3,5,5-trimethyl-hexan-1-ol; 2,6,10,10-tetramethyl-1-oxa-spiro[4.5]decan-6-ol; 5-isopropyl-2-methyl-cyclohexanol: 4-isopropyl-1-methyl-cyclohex-3-enol; 6,6-dimethyl-2-methylene-bicyclo[3.1.1]heptan-3-ol; 4,6,6-trimethyl-bicyclo[3.1.1]hept-3-en-2-ol; 4-hydroxymethyl-2-methoxy-phenol; 2-(2,2,3-trimethyl-cyclopent-3-enyl)-ethanol; 2-(5-methyl-5-vinyl-tetrahydro-furan-2-yl)-propan-2-ol; 3,3,5-trimethyl-cyclohexanol; 3-hydroxy-4-phenyl-butan-2-one; 2-(1-hydroxy-1-methyl-ethyl)-5-methyl-cyclohexanol; 3,7-dimethylocta-1,6-dien-3-ol; 3,7-dimethyl-6-octenol; methyl 2-hydroxybenzoate; ethyl 2-hydroxybenzoate; exo-1,7,7-trimethylbicyclo[2.2.1]heptan-2-ol; 2-ethyl-1,3,3-trimethyl-bicyclo[2.2.1]heptan-2-ol; 1-octanol; 2-octanol; 3-octanol; 4-octanol; 1-nonanol; 2-methoxy-4-prop-1-enyl)phenol and 6,6-dimethyl-bicyclo[3.1.1]hept-2-ene-2-methanol.

Further examples of organoleptic alcohols R¹—OH/R³—OH from which the residues Y and X respectively are derived are described, for example, in S. Arctander Perfume and Flavor Chemicals Vols. 1 and 2, Arctander, Monclair, N.J. USA 1989, which is incorporated by reference.

Alcohols such as methyl 2-hydroxycyclohexanecarboxylate are not known to have organoleptic properties and thus would not fall within the definition of organoleptic alcohols.

Examples of alkanols R²—OH are: ethanol, propanol, propylene glycol, glycerol, sorbitol, xylitol, lactic acid, alpha-glucose and ascorbic acid.

Particular embodiments are compounds of formula (I) wherein both, X and Y are the residue of an organoleptic alcohol. Examples for such compounds are methyl 2-((2E)-3-(((Z)-hex-3-enyloxy)carbonyl)acryloyloxy)benzoate, (Z)-hex-3-enyl 2-methyl-4-oxo-4H-pyran-3-yl fumarate, and 2-ethoxy-4-formylphenyl (Z)-hex-3-enyl fumarate and (Z)-hex-3-enyl 2-methoxy-4-(3-oxobutyl)phenyl fumarate.

Further particular embodiments are compounds of formula (I) wherein X is the residue of ethanol, i.e. X is CH₃—CH₂—O and Y is the residue R³—O of an organoleptic alcohol R³—OH selected from 4-allyl-2-methoxy-phenol and 2-isopropyl-5-methyl-phenol; compounds of formula (I) wherein X is the residue of an alkanol selected from propylene glycol and lactic acid and Y is the residue R³—O of an organoleptic alcohol R³—OH selected from 2-isopropyl-5-methylcyclohexanol, 1,7,7-trimethyl-bicyclo[2.2.1]heptan-2-ol, 4-allyl-2-methoxy-phenol, 2-isopropenyl-5-methylcyclohexan-1-ol, 2-isopropyl-5-methyl-phenol and 6,6-dimethyl-2-methylene-bicyclo[3.1.1]heptan-3-ol; compounds of formula (I) wherein X is the residue of sorbitol, e.g. X is —O—CH₂—(CH(OH))₄—CH₂OH, and Y is the residue R³—O of an organoleptic alcohol R³—OH selected from 2-isopropyl-5-methylcyclohexanol, 1,7,7-trimethyl-bicyclo[2.2.1]heptan-2-ol, 4-allyl-2-methoxy-phenol, 2-isopropenyl-5-methylcyclohexan-1-ol, 2-isopropyl-5-methyl-phenol and 6,6-dimethyl-2-methylene-bicyclo[3.1.1]heptan-3-ol; compounds of formula (I) wherein X is the residue of glycerol, e.g. X is —O—CH₂—CH(OH)—CH₂OH, and Y is the residue R³—O of an organoleptic alcohol R³—OH selected from 2-isopropyl-5-methylcyclohexanol, 1,7,7-trimethyl-bicyclo[2.2.1]heptan-2-ol, 4-allyl-2-methoxy-phenol, 2-isopropenyl-5-methylcyclohexan-1-ol, 2-isopropyl-5-methyl-phenol and 6,6-dimethyl-2-methylene-bicyclo[3.1.1]heptan-3-ol; and compounds of formula (I) wherein X is the residue of ascorbic acid, e.g. X is

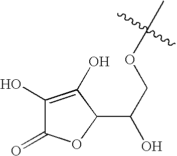

, and Y is the residue R³—O of an organoleptic alcohol R³—OH selected from 2-isopropyl-5-methylcyclohexanol, 1,7,7-trimethyl-bicyclo[2.2.1]heptan-2-ol, 4-allyl-2-methoxy-phenol, 2-isopropenyl-5-methylcyclohexan-1-ol, 2-isopropyl-5-methyl-phenol and 6,6-dimethyl-2-methylene-bicyclo[3.1.1]heptan-3-ol.

In a specific embodiment of the invention the oral composition comprises a compound selected from the list consisting of 2,3-dihydroxypropyl 2-isopropyl-5-methylcyclohexyl fumarate (1), ethyl 2-methyl-4-oxo-4H-pyran-3-yl fumarate (2), 2-ethoxy-4-formylphenyl ethyl fumarate (3), methyl 2-((E)-3-(ethoxycarbonyl)acryloyloxy)benzoate (4), 2,3,4,5,6-pentahydroxyhexyl 2-isopropyl-5-methylcyclohexyl fumarate (5), cinnamyl ethyl fumarate (6) and ethyl (Z)-hex-3-enyl fumarate (7).

The compounds of formula (I) are essentially odourless, but when applied to the oral cavity, they chemically bind the VSCs and subsequently undergo a transformation in which the organoleptic alcohol is released by ester hydrolysis catalysed by the esterases present in saliva. This newly-formed organoleptic compound serves as a masking agent and, depending on the nature of the released compound, may also serve as an antibacterial agent. Organoleptic compounds having the capability of acting as an odour masking agent and as an antibacterial are, for example, methyl salicylate (ethyl 2-hydroxybenzoate), menthol (2-isopropyl-5-methylcyclohexanol), isoeugenol ((2-methoxy-4-prop-1-enyl)phenol) and thymol (2-isopropyl-5-methyl-phenol). These compounds often have a rather harsh taste when applied directly to the oral cavity. Thus, a controlled release of such compounds over a longer period, as provided by the compounds of formula (I), would be desirable.

The term “oral composition” as used herein refers to food and non-food compositions which are designed to be taken into the mouth and thus come into contact with saliva. Such compositions include chewing gum, candies, edible films, in particular breath strips, and beverages. In a particular embodiment the term “oral composition” refers to compositions which are suitable for oral hygiene such as chewing gum and oral care products, for example, toothpaste, mouthwash, mouth spray and gargle compositions, candies, lozenges, pastilles, and the like.

Breath strips are edible films which are placed in the oral cavity to administer thereto an active agent such as a flavourant or breath-freshening agent.

The oral composition according to the present invention comprises an effective amount of at least one compound of formula (I) as hereinabove defined. For example, the oral composition according to the present invention comprises about 0.05 weight % to about 2 weight %, for example about 0.4 weight % to about 1 weight %, of at least one compound of formula (I) based on the total weight of the oral composition.

Oral compositions may comprise additional ingredients and excipients well known in the art, in particular flavour ingredients for providing a desired flavour accord and/or cooling agents for providing a fresh mouth feel. Examples of known flavour ingredients and cooling agents may be found in one of the FEMA (Flavour and Extracts Manufacturers Association of the United States) publications or a compilation thereof which is available from and published by FEMA and contains all FEMA GRAS (Generally Regarded As Safe) publications, 1965-present, in particular publications GRAS 1-21 (the most recent one being GRAS 21 published 2003), or in Allured's Flavor and Fragrance Materials 2004, published by Allured Publishing Inc. Examples of known excipients for oral care products may also be found in Gaffar, Abdul, Advanced Technology, Corporate Technology, Department of Oral Care, Colgate-Palmolive Company, Piscataway, N.J., USA. Editor(s): Barel, Andre O.; Paye, Marc; Maibach, Howard I., Handbook of Cosmetic Science and Technology (2001), p. 619-643. Publisher: Marcel Dekker, Inc., New York, N.Y., and in Cosmetics: Science and technology, 2nd edition, p. 423-563. Edited by M. S. Balsam and E. Sagarin, Wiley Interscience, 1972.

Particular examples of cooling agents may include, but are not limited to, menthol, menthone, isopulegol, N-ethyl p-menthanecarboxamide (WS-3), N,2,3-trimethyl-2-isopropylbutanamide (WS-23), menthyl lactate, menthone glycerine acetal (Frescolat® MGA), mono-menthyl succinate (Physcool®), mono-menthyl glutarate, O-menthyl glycerine (CoolAct® 10), 2-sec-butylcyclohexanone (Freskomenthe®) and 2-isopropyl-5-methyl-cyclohexanecarboxylic acid (2-pyridin-2-yl-ethyl)-amide. Further examples of cooling agents can be found e.g. in WO 2006/125334 and WO 2005/049553, which are incorporated by reference.

As an example, the composition for toothpaste may comprise in addition to the active ingredient, i.e. compound(s) of formula (I), other compounds commonly used in toothpaste, such as oral disinfectant, abrasive, humectant, detergent, binder, frothing agent, sweetening agent, preservative, buffering agent, flavours and cooling agents and may be prepared following the procedures known to the skilled person.

According to the inventors best knowledge, the compounds of formula (I) have never been described in the literature and thus are novel in their own right. Accordingly, the present invention refers in a further aspect to compounds of formula (I) as hereinabove defined.

The compounds of the present invention may be prepared by known procedures for the preparation of symmetrical and unsymmetrical fumaric acid diesters respectively. For compounds of the present invention wherein X is the residue of ethanol, i.e. wherein R² is ethyl, (E)-ethyl 3-(chlorocarbonyl)acrylate is reacted with an organoleptic alcohol Y—H, wherein Y has the same meaning as given above, in a standard esterification reaction.

Compounds of formula (I) wherein X is other than a residue of ethanol may be prepared according to the general procedure outlined below in Scheme 1, Y and X have the same meaning as given above.

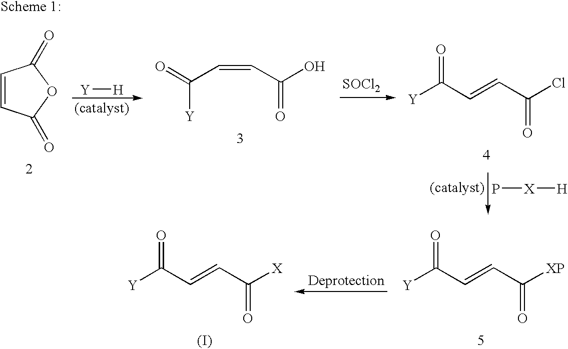

Maleic anhydride 2 is opened with either X—H or Y—H by a thermal reaction or in the presence of a catalyst. The resulting maleic acid monoester 3 is then reacted with thionyl chloride or a similar chlorinating reagent, which converts the free carboxyl group to the acid chloride under concomitant E/Z-isomerization of the double bond, yielding the corresponding (E)-3-(chlorcarbonyl)acrylic acid ester 4. This acid chloride is then esterified with Y—H when maleic anhydride is opened with X—H and esterified with X—H when maleic anhydride is opened with Y—H. If X—H is a diol, triol or polyol, the nonreacting hydroxyl group(s) may optionally be protected by protective group(s) P, such as acetals, ketals, ethers or silyl ethers, which are then removed in the final deprotection step (Scheme 1), such as the acid-catalyzed cleavage of an acetal or ketal moiety, the fluoride mediated cleavage of a silyl ether group, or the removal of labile ether groups according to the procedure known to the person skilled in the art.

Instead of the esterification in step three with a single compound Y—H or X—H, for example a mint oil, comprising a mixture of organoleptic alcohols, such as menthol, neomenthol, isopulegol, neoisomenthol, and lavandulol, may be added, to give a mixture of compounds of formula (I), which in turn when applied to the oral cavity, may release the individual organoleptic alcohols in similar proportions as present in the mint oil.

Alternatively, a fumaric acid monoester 6 might be prepared by methods known to the person skilled in the art, which will be esterified with X—H as show in Scheme 2 (Y and X have the same meaning as given above). The esterification step leading to compound of formula (I) may be carried out by using biocatalysts such as a lipase.

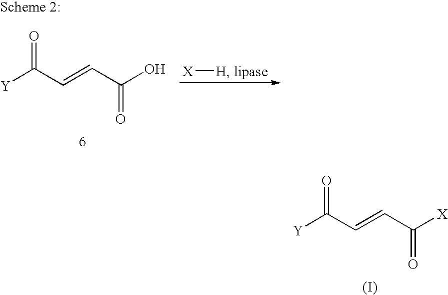

The compositions and methods are now further described with reference to the following non-limiting examples. These examples are for the purpose of illustration only and it is understood that variations and modifications can be made by one skilled in the art without departing from the scope of the invention. It should be understood that the embodiments described are not only in the alternative, but can be combined.

### EXAMPLE 1

### 2,3-Dihydroxypropyl 2-isopropyl-5-methylcyclohexyl fumarate (1)

a) The mixture of (−)-menthol (165.6 g, 1.1 mol) and maleic anhydride (98.0 g, 1.0 mol) is heated to 10° C. during 3 h, then cooled to room temperature and diluted with MTBE (400 ml). The product is extracted with sat. aq. NaHCO₃-solution (1.1 l, pH=8), and the aq. solution washed with 2 portions of MTBE (each 100 ml). Ice is added to the aq. solution before acidification with conc. aq. HCl-solution (152 g). Extraction with MTBE, washing with brine, drying over MgSO₄ and removal of the solvent yields (Z)-3-((2-isopropyl-5-methylcyclohexyloxy)carbonyl)acrylic acid (265 g) as a white crystalline product, which is dissolved in cyclohexane (600 ml). N,N′-dimethylformamide (DMF, 20.8 ml, 0.27 mol) is added and the solution warmed to 70° C. At this temperature, thionylchloride (65.3 ml, 0.9 mol) is added dropwise during 30 min. The temperature rises to 80° C. and is maintained there with external heating for 1.5 h. The heating bath is removed and the solvent evaporated in a rotary vaporizer (RV) at 54° C./30 mbar, followed by drying of the residue at 50° C./0.25 mbar for 2 h. (E)-2-Isopropyl-5-methylcyclohexyl 3-(chlorocarbonyl)acrylate is obtained as a brownish oil (254.5 g, 93%), containing traces of residual DMF (ca. 5%).

IR: 1766 m, 1719 vs, 1456 w, 1269 vs, 1177 s, 1097 m, 971 m, 951 m, 668 w, 645 m.

¹H-NMR: 6.95 (d, J=2.0 Hz, 2H), 4.80 (td, J=10.9, 4.4 Hz, 1H), 1.95-2.04 (m, 1H), 1.78-1.88 (m, 1H), 1.65-1.72 (m, 2H), 1.40-1.52 (m, 2H), 0.98-1.09 (m, 2H), 0.90 (t, J=6.5 Hz, 6H), 0.84-0.93 (m, 1H), 0.75 (d, J=6.8 Hz, 3H).

¹³C-NMR: 165.4 (s), 163.3 (s), 138.4 (d), 136.5 (d), 76.3 (d), 46.9 (d), 40.6 (t), 34.1 (t), 31.4 (d), 26.3 (d), 23.3 (t), 21.9 (q), 20.7 (q), 16.2 (q).

MS: 237 (1), 138 (59), 123 (45), 96 (23), 95 (100), 83 (161), 82 (34), 81 (74), 55 (27), 43 (17), 41 (22).

b) The solution of DL-α,β-isopropylidenglycerin (123.0 g, 0.93 ml) and tributylamine (176.0 g, 0.95 mol) in MTBE (300 ml) is cooled with an icebath and the solution of (E)-2-isopropyl-5-methylcyclohexyl 3-(chlorocarbonyl)acrylate (254.0 g, 0.93 mol) in MTBE (100 ml) is added dropwise during 40 min. (internal temperature 23-25° C.). After 30 min. additional stirring, water is added (100 ml), followed by 2 N aq. HCl-solution (40 ml). The aqueous layer is separated and the organic layer is washed twice with 2 N aq. HCl-solution (each 25 ml), water and brine. After drying over MgSO₄ and evaporation of the solvents i.RV and drying of the residue at 55° C./0.1 mbar during 30 min., but-2-enedioic acid 2,2-dimethyl-[1,3]dioxolan-4-ylmethyl ester 2-isopropyl-5-methyl-cyclohexyl ester is obtained as brownish oil (312.0 g, 91%).

IR: 1717 vs, 1644 w, 1293 s, 1256 vs, 1149 vs, 841 m.

¹H-NMR: 6.83 (s, 2H), 4.75 (td, J=10.9, 4.3, 1H), 4.29-4.38 (m, 1H), 4.22-4.28 (m, 1H), 4.13-4.21 (m, 1H), 4.07 (dd, J=8.6, 6.6 Hz, 1H), 1.94-2.02 (m, 1H), 1.76-1.87 (m, 1H), 1.61-1.70 (m, 2H), 1.40 (s, 3H), 1.35-1.53 (m, 2H), 1.33 (s, 3H), 0.93-1.10 (m, 2H), 0.87 (dd, J=6.9 Hz, 6H), 0.82-0.91 (m, 2H), 0.72 (d, J=7.1 Hz, 3H).

¹³C-NMR: 164.7 (s), 164.3 (s), 134.8 (d), 132.5 (d), 109.9 (s), 75.4 (d), 73.3 (d), 66.2 (t), 65.5 (t), 46.9 (d), 40.6 (t), 34.1 (t), 31.3 (d), 26.6 (q), 26.2 (d), 25.3 (q), 23.4 (q), 21.9 (t), 20.6 (q), 16.3 (q).

MS: 353 (70, [M−CH₃]⁺), 138 (74), 101 (70), 99 (69), 95 (100), 82 (44), 81 (69), 57 (42), 55 (64), 43 (81).

c) The mixture of glycerol (66 g), boric acid (0.94 g, 165 mmol), water (6.6 g) and but-2-enedioic acid 2,2-dimethyl-[1,3]dioxolan-4-ylmethyl ester 2-isopropyl-5-methyl-cyclohexyl ester (22.1 g, 60 mmol) is heated to 100° C. during 18 h under intense stirring. While still hot, the glycerol phase is separated and removed and the supernatant is washed with hot glycerol/water 3:2 (10 ml). 2,3-Dihydroxypropyl 2-isopropyl-5-methylcyclohexyl fumarate is obtained as a viscous, yellowish and slightly turbid oil (17.3, 88%).

IR: 3434 br., 1716 vs, 1293 vs, 1256 vs, 1157 s, 772 m.

¹H-NMR: 6.87 (d, J=2.0, 2H), 4.79 (td, J=10.9, 4.4, 1H), 4.23-4.33 (m, 2H), 3.96-4.04 (m, 1H), 3.73 (dd, J=11.5, 3.9, 1H), 3.63 (dd, J=11.3, 6.1, 1H), 3.40 (s, 1H), 1.98-2.04 (m, 1H), 1.80-1.91 (m, 1H), 1.66-1.75 (m, 2H), 1.39-1.58 (m, 2H), 0.98-1.15 (m, 2H), 0.91 (dd, J=7.8, 6.8, 6H), 0.76 (d, J=6.8, 3H).

¹³C-NMR: 171.3 (s), 165.1 (s), 164.3 (s), 134.7 (d), 132.5 (d), 75.5 (d), 69.8 (d), 65.8 (t), 63.2 (t), 60.4 (t), 46.8 (d), 40.5 (t), 34.0 (t), 31.3 (d), 26.1 (d), 23.3 (t), 21.8 (q), 20.9 (q), 20.6 (q), 16.2 (q), 14.0 (q).

MS: 310 (<1, [M−H₂O]⁺), 297 (4), 237 (6), 191 (4), 173 (9), 156 (5), 139 (25), 138 (70), 123 (36), 99 (51), 95 (100), 81 (73), 55 (48).

### EXAMPLE 2

### Ethyl 2-methyl-4-oxo-4H-pyran-3-yl fumarate (2)

a) Fumaric acid monoethyl ester (43.24 g, 0.30 mol) is suspended in 1,2-dichloroethane (50 ml) and DMF (2.0 ml) is added. The mixture is vigorously stirred while freshly distilled SOCl₂ is added dropwise during 20 min. The resulting mixture is heated to 70° C. for 1 h, than to 80° C. for 1 h. After cooling to room temperature, the solvent is removed by distillation at ambient pressure. Vacuum is applied (15 mbar) and 3-chlorocarbonyl-acrylic acid ethyl ester is distilled at 77-80° C. as a colourless liquid (37.37 g, 77%).

IR: 1765 m, 1721 vs, 1302 s, 1260 s, 1182 s, 1096 s, 1015 s, 969 s, 863 w, 806 w, 733 w, 666 w, 633 m.

¹H-NMR: 6.97, 6.90 (AB, J_{AB}=15.4, 2H), 4.26 (q, J=7.2 Hz, 2H), 1.30 (t, J=7.2 Hz, 3H).

¹³C-NMR: 165.3 (s), 163.6 (s), 137.8 (d), 136.6 (d), 62.0 (t), 13.9 (q).

MS: 127 (100, [M−Cl]⁺), 117 (34), 64 (99), 89 (58), 82 (38), 71 (10), 54 (34).

b) Maltol (9.35 g, 74 mmol, 1.05 equiv.), pyridine (9.8 ml, 120 mmol, 1.7 equiv.) and 4-dimethylaminopyridine (112 mg) are suspended in methyl t-butylether (MTBE, 100 ml) and the suspension is cooled with an icebath. A solution of 3-chlorocarbonyl-acrylic acid ethyl ester (11.29 g, 70 mmol) in MTBE (30 ml) is added dropwise during 20 min. The resulting suspension is stirred for 30 min. at 3° C., than for 2.5 h at room temperature. The mixture is hydrolyzed with ice/2N aq. HCl and extracted with EtOAc. The organic layer is washed with 0.5 N aq. HCl-solution, then twice with brine and dried over MgSO₄. The crude obtained after removal of the solvents is purified via FC on SiO₂ (hexane/EtOAc 1:4) to isolate ethyl 2-methyl-4-oxo-4H-pyran-3-yl fumarate as a viscous, red-brown oil (8.95 g, 51%).

IR: 1753 m, 1721 s, 1659 vs, 1643 vs, 1421 m, 1292 s, 1240 s, 1161 vs, 1133 vs, 1029 m, 976 m, 831 m.

¹H-NMR: 7.94 (d, J=5.8, 1H), 6.63 (d, J=5.8, 1H), 4.50 (q, J=7.1, 2H), 2.49 (s, 3H), 1.54 (t, J=7.1, 3H).

¹³C-NMR: 171.23 (s), 164.26 (s), 161.36 (s), 159.05 (s), 154.27 (d), 138.22 (s), 136.08 (d), 131.12 (d), 116.66 (d), 61.42 (t), 14.84 (q), 13.94 (q).

MS: 253 (1, [M+H]⁺), 224 (4), 207 (16), 179 (5), 154 (8), 137 (8), 127 (100), 126 (18), 99 (23), 55 (22).

### EXAMPLE 3

### 2-Ethoxy-4-formylphenyl ethyl fumarate (3)

The procedure described in Example 2b is repeated with ethylvanillin (8.63 g, 52 mmol), pyridine (6.4 ml, 80 mmol, 1.5 equiv.), 4-dimethylaminopyridine (80 mg) and 3-chlorocarbonyl-acrylic acid ethyl ester (8.45 g, 70 mmol) in toluene (90 ml). The crude is purified via FC on SiO₂ (hexane/EtOAc 5:1) to isolate 2-ethoxy-4-formylphenyl ethyl fumarate as a viscous, pale yellow oil (10.07 g, 66%).

IR: 1749 m, 1722 vs, 1696 vs, 1599 m, 1501 m, 1434 m, 1288 vs, 1261 vs, 1115 vs, 1033 vs, 974 m, 671 m.

¹H-NMR: 9.94 (s, 1H), 7.46-7.50 (m, 2H), 7.26 (d, J=7.8, 1H), 7.07 (d, J=1.3, 2H), 4.31 (q, J=7.1, 2H), 4.13 (q, J=6.9, 2H), 1.39 (t, J=6.4, 3H), 1.35 (t, J=6.6, 3H).

¹³C-NMR: 190.8 (d), 164.5 (s), 162.1 (s), 151.0 (s), 144.4 (s), 135.6 (d), 135.3 (s), 131.8 (d), 124.2 (d), 123.0 (d), 111.8 (d), 64.6 (t), 61.5 (t), 14.4 (q), 14.0 (q).

MS: 292 (2, M⁺), 247 (3), 219 (1), 166 (7), 137 (10), 127 (100), 109 (5), 99 (27), 81 (11), 55 (19).

### EXAMPLE 4

### Methyl 2-((E)-3-(ethoxycarbonyl)acryloyloxy)benzoate (4)

The procedure described in Example 2b is repeated with methyl salicylate (11.0 g, 72 mmol), pyridine (9.2 g, 116 mmol, 1.7 equiv.), 4-dimethylaminopyridine (100 mg) and 3-chlorocarbonyl-acrylic acid ethyl ester (11.1 g, 68 mmol) in MTBE (100 ml). The crude is purified via FC on SiO₂ (hexane/MTBE 10:1→5:1→1:1) to isolate methyl 2-((E)-3-(ethoxycarbonyl)acryloyloxy)benzoate as a viscous, pale yellow oil (12.9 g, 68%).

IR: 1750 m, 1718 vs 1607 w, 1291 vs, 1256 vs, 1200 vs, 1139 vs, 1081 vs, 1028 m, 756 m, 735 m, 700 m, 674 m.

¹H-NMR: 7.99 (dd, J=7.7, 1.6, 1H), 7.53 (td, J=7.8, 1.8, 1H), 7.29 (td, J=7.6, 1.1, 1H), 7.08-7.11 (m, 1H), 7.03 (d, J=6.1, 2H), 4.24 (q, J=7.1, 2H), 3.77 (s, 3H), 1.28 (t, J=7.1, 3H).

¹³C-NMR: 164.5 (s), 164.4 (s), 163.4 (s), 149.9 (d), 135.2 (d), 133.8 (d), 132.3 (d), 131.7 (d), 126.2 (d), 123.4 (d), 122.8 (s), 61.3 (t), 52.1 (q), 13.9 (q).

MS: 278 (<1, [M−OH]⁺), 247 (22), 233 (3), 152 (7), 127 (100), 120 (18), 113 (7), 99 (18), 92 (13), 82 (6), 71 (7), 55 (17).

### EXAMPLE 5

### 2,3,4,5,6-Pentahydroxyhexyl 2-isopropyl-5-methylcyclohexyl fumarate (5)

a) (Z)-3-((2-isopropyl-5-methylcyclohexyloxy)carbonyl)acrylic acid (25 g, 0.10 mol) is heated with fumaryl chloride (0.35 g, 2 mol %) to 100° C. during 5 h. The mixture is cooled to room temperature, poured on water and extracted with MTBE. The organic layer is separated, dried over MgSO₄ and purified by FC over SiO₂ (hexane/MTBE 10:1→5:1→EtOAc 100%). (E)-3-((2-Isopropyl-5-methylcyclohexyloxy)carbonyl)acrylic acid is isolated as a colourless, viscous oil (21.5 g, 86%).

IR: 3500-3000 br., 1703 vs, 1644 m, 1260 vs, 1010 s, 653 m.

¹H-NMR: 11.73 (br., 1H), 6.89 (d, J=15.9 Hz, 1H), 6.79 (d, J=15.9 Hz, 1H), 4.73-4.84 (m, 1H), 1.96-2.02 (m, 1H), 1.77-1.87 (m, 1H), 1.61-1.71 (m, 2H), 1.37-1.49 (m, 2H), 0.95-1.06 (m, 2H), 0.90-0.82 (m, 1H), 0.86 (t, J=7.1 Hz, 6H), 0.72 (d, J=7.1 Hz, 3H).

¹³C-NMR: 170.0 (s), 164.2 (s), 136.1 (d), 132.4 (d), 75.7 (d), 46.9 (d), 40.6 (t), 34.0 (t), 31.3 (d), 26.2 (d), 23.3 (t), 21.9 (q), 20.6 (q), 16.2 (q).

MS: 237 (<1, [M−OH]⁺), 138 (42), 123 (36), 99 (58), 95 (100), 80 (81).

b) To the solution of D-sorbitol (1.82 g, 10 mmol), DMAP (1.60 g, 13 mmol) and dicyclohexyl carbodiimide (5.36 g, 26 mmol) in DMF (50 ml) is added the solution of (E)-3-((2-isopropyl-5-methylcyclohexyloxy)carbonyl)acrylic acid (5.08 g, 20 mmol) in DMF (20 ml). The mixture is stirred for 3 days at room temperature, and then filtered. The filtrate is poured on 5% aq. HCl-solution and extracted with EtOAc. The organic layer is washed with brine and dried over MgSO₄. The crude is purified via FC on SiO₂ (hexane/EtOAc 10:1→5:1→1:1). Besides some dimenthyl fumarate, fractions with sorbitol-(E)-3-((2-isopropyl-5-methylcyclohexyloxy)carbonyl)acrylic acid di- and triesters are isolated. From the most polar fractions, 2,3,4,5,6-pentahydroxyhexyl 2-isopropyl-5-methylcyclohexyl fumarate is isolated (1.7 g, 35%).

Mixture of 2 regioisomers.

IR: 3364 br., 1715 s, 1656 vs, 1294 s, 1257 s, 1158 m, 662 m.

¹H-NMR: 6.80 (d, J=2H), 4.66-4.76 (m, 1H), 4.54 (series of m, 7H), 3.51-4.13 (m, 7H), 1.89-2.00 (m, 1H), 1.72-1.86 (2 m, 2H), 1.56-1.69 (m, 2H), 1.31-1.51 (m, 2H), 0.84 (dd, J=9.1, 6.8 Hz, 6H), 0.68 (d, J=6.8 Hz, 3H).

¹³C-NMR: 165.4 (s), 165.2 (s), 164.6 (s), 164.6 (s), 134.6 (d), 134.5 (d), 133.0 (d), 132.9 (d), 73.5 (d), 73.1 (d), 72.2 (d), 71.8 (d), 71.5 (d), 69.7 (d), 69.6 (d), 69.5 (d), 67.0 (t), 66.5 (t), 63.9 (t), 63.5 (t), 46.9 (d), 40.6 (t), 34.1 (t), 31.4 (q), 31.3 (d), 26.2 (d), 23.3 (t), 21.9 (q), 20.7 (q), 16.3 (q).

MS (APCI pos. +NH₄OAc): 436 (100, [M+NH₄]⁺), 419 (25, [M++]⁺).

### EXAMPLE 6

### Cinnamyl Ethyl Fumarate (6)

The procedure described in Example 2b is repeated with cinnamic alcohol (11.4 g, 85 mmol), pyridine (10.8 g, 140 mmol, 1.7 equiv.), 4-dimethylaminopyridine (100 mg) and 3-chlorocarbonyl-acrylic acid ethyl ester (13.5 g, 80 mmol) in MTBE (100 ml). The crude is purified via flash chromatography (FC) on SiO₂ (hexane/MTBE 10:1→5:1) to isolate cinnamyl ethyl fumarate as a colourless oil (14.5 g, 73%).

IR: 1716 s, 1645 w, 1448 w, 1368 w, 1289 vs, 1255 vs, 1222 m, 1149 vs, 1028 m, 964 vs, 774 m, 743 m, 691 s.

¹H-NMR: 7.33-7.37 (m, 2H), 7.25-7.31 (m, 2H), 7.19-7.25 (m, 1H), 6.88 (s, 2H), 6.64 (d, J=15.9 Hz, 1H), 6.26 (dt, J=15.9, 6.4 Hz, 1H), 4.80 (dd, J=6.4, 1.4 Hz, 2H), 4.21 (q, J=7.1 Hz, 2H), 1.26 (t, J=7.1 Hz, 3H).

¹³C-NMR: 164.4 (s), 164.2 (s), 135.7 (s), 134.3 (d), 133.6 (d), 132.9 (d), 128.3 (d), 127.8 (d), 126.3 (d), 122.1 (d), 65.4 (t), 60.9 (t), 13.7 (q).

MS: 260 (7, M⁺), 214 (2, [M−EtOH]⁺), 186 (5), 169 (4), 143 (5), 133 (50), 128 (68), 127 (72), 117 (89), 115 (100), 105 (54), 99 (33), 91 (25), 77 (15), 55 (17).

### EXAMPLE 7

### Ethyl (Z)-hex-3-enyl fumarate (7)

The procedure described in Example 2b is repeated with Z-3-hexenol (1.44 g, 14 mmol), pyridine (2.3 ml, 28 mmol, 2.0 equiv.), 4-dimethylaminopyridine (37 mg) and 3-chlorocarbonyl-acrylic acid ethyl ester (2.28 g, 14 mmol) in MTBE (40 ml). The crude is purified via FC on SiO₂ (hexane/MTBE 19:1) to isolate ethyl (Z)-hex-3-enyl fumarate as a colourless oil (2.70 g, 85%).

IR: 1720 s, 1647 w, 1294 s, 1256 s, 152 vs, 1029 m, 988 m, 774 w.

¹H-NMR: 6.80 (s, 2H), 5.45-5.52 (m, 1H), 5.24-5.32 (m, 1H), 4.22 (q, J=7.1 Hz, 2H), 4.16 (t, J=6.8 Hz, 2H), 2.36-2.42 (m, 2H), 1.98-2.06 (m, J=7.5, 7.5, 7.5, 7.5, 1.5 Hz, 2H), 1.28 (t, J=7.2 Hz, 3H), 0.93 (t, J=7.6 Hz, 3H).

¹³C-NMR: 164.9 (s), 164.8 (s), 134.8 (d), 133.6 (d), 133.4 (d), 123.2 (d), 64.7 (t), 61.2 (t), 26.5 (t), 20.5 (t), 14.1 (q), 14.0 (q).

MS: 226 (<1, M⁺), 208 (<1, [M−H₂O]⁺), 181 (<1), 145 (<1), 127 (27), 99 (14), 82 (100), 67 (97), 55 (26), 41 (21).

### EXAMPLE 8

### Reduction of Methanethiol (MeSH) in Headspace

The compounds listed in Table 1 are dissolved to a final concentration of 100 μM, 200 μM and 500 μM in 1 ml of phosphate buffer at pH 7 in a closed GC-headspace vial. MeSH is added to a final concentration of 100 μM and the mixture is equilibrated for 1 h. Samples are heated to 75° C. and 1 ml of the headspace above the reaction mixture is injected onto a column suitable for separation of sulphur compounds (SPW1-sulfur, Supelco). The temperature program is set to 1 min initial temperature at 50° C., heating at a rate of 10° C./min to 100° C. and further heating at 20° C./min to 200° C. The headspace level of MeSH is compared to a blank sample, i.e. a sample without the active compound. The results are given in Table 1 below.

TABLE 1
% reduction of MeSH levels in the headspace

| | | % reduction | | |
|---|---|---|---|---|
| | | of MeSH in the headspace | | |
| Compound No. | CLogP | 500 μM | 200 μM | 100 μM |
| 1 (Ex. 1) | 3.15 | >90 | 73.7 | 81.5 |
| 2 (Ex. 2) | 0.88 | >90 | >92 | >92 |
| 3 (Ex. 3) | 2.40 | >90 | >92 | >90 |
| 4 (Ex. 4) | 2.47 | >90 | >92 | >90 |
| 5 (Ex. 5) | 2.64 | >90 | n.d. | 71.6 |
| 6 (Ex. 6) | 3.68 | 85 | n.d. | 45.8 |
| 7 (Ex. 7) | 3.47 | 54.1 | 39.2 | 33 |
| Dihexyl fumarate (A) | 6.07 | 10.9 | 1.9 | 4.3 |
| 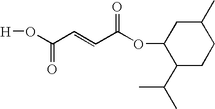 | 4.27 | 0 | 1.7 | 11.9 |
| (B) | | | | |

As can be seen from the results above only the compounds wherein the fumarate moiety is esterified on both sides, and with a sufficient hydrophilicity, i.e. CLogP≦4.5, have a good ability to bind MeSH in an aqueous environment and thereby reduce its level in the headspace. Double esterified compounds with high CLogP, see compound (A), show only a very low reactivity towards MeSH in an aqueous environment. Mono-esterified compounds such as compound (B) also have a low reactivity.

### EXAMPLE 9

### Reduction of Allyl Mercaptan

The compounds given in Table 2 are dissolved in DMSO to a final concentration of 100 mM and serially diluted in the same solvent. Aliquots of the solutions of different active compounds (2.5 μl) are distributed to individual wells of a microtiter plate. 100 μl of a 200 μM allyl mercaptan-solution (in 50 mM phosphate buffer, pH 7) are added to each well and the plates are immediately sealed. After 15 min of incubation, the unreacted allyl mercaptan is derivatised by adding to each well of the microtiter plate 100 μl of a monobromobimane (obtained from Fluka, Buchs, Switzerland) stock solution (0.5 mM in 1 M NaCO3, pH 8.8). After 10 min the fluorescence in the wells of the microtiter plates is measured on a Flex-station (Molecular devices, Sunnyvale, Calif., USA) with an excitation wavelength of 385 nm and an emission wavelength of 480 nm. After the fluorescence determination, from all the wells the blank value containing only buffer and DMSO is subtracted. The fluorescence of control wells with allyl mercaptan and DMSO only is then compared to the fluorescence in wells containing potential allyl mercaptan trapping agents (compound 1 to 5) to calculate the inhibition in percent. Table 2 lists the results obtained.

TABLE 2
Reduction (%) of allyl mercaptan by different doses of the
active compounds

| | Test concentration [μM] | | | | | |
|---|---|---|---|---|---|---|
| | 1000 | 500 | 250 | 125 | 62.5 | 31.25 |
| Comp. No. | % reduction of allyl mercaptan | | | | | |
| 1 (Ex. 1) | 86.0 | 68.8 | 43.2 | 22.5 | 9.9 | 0.1 |
| 2 (Ex. 2) | 100.0 | 100.0 | 98.4 | 65.3 | 31.9 | 11.1 |
| 3 (Ex. 3) | 100.0 | 100.0 | 99.8 | 74.5 | 32.7 | 12.8 |
| 4 (Ex. 4) | 100.0 | 99.8 | 95.3 | 67.6 | 32.3 | 14.4 |
| 5 (Ex. 5) | 89.3 | 64.4 | 38.4 | 19.3 | 8.1 | 1.0 |

As can be seen from the results above the compounds of the present invention have the ability to react at equimolar concentration with allyl mercaptan even at a very low test concentration, and are therefore useful for consumer products to prevent bad breath, for example after the consumption of a garlic containing meal.

### EXAMPLE 10

### Reduction of Methanethiol (MeSH) in Saliva

The compounds given in Table 3 are dissolved in GC-headspace vials in saliva donations pooled form four donors at a concentration of 500 μM. After a conditioning period of 1 and 2.5 h respectively, MeSH is added at a concentration of 200 μM, and 1 hour later MeSH level in the headspace is determined as described in Example 8. The results are given in Table 3 below.

TABLE 3
Reduction of MeSH (%)

| Compound | after 1 h | after 2½ h |
|---|---|---|
| 1 (Ex. 1) | 72.7 | 40.5 |
| 2 (Ex. 2) | 75.9 | 32.5 |
| 3 (Ex. 3) | 36.2 | 73.3 |
| 4 (Ex. 4) | 28.1 | 74.3 |
| 5 (Ex. 5) | 48.2 | 75.1 |

Although the compounds of the present invention are meta-stabile and are cleaved by salivary enzymes as can bee seen from Example 11, they are sufficiently stable to reduce volatile sulphur compounds for a sufficiently long period of time.

### EXAMPLE 11

### Release of Organoleptic Compounds by Cleavage in the Presence of Saliva

The substrates, i.e. compounds according to the present invention, given in Table 4 are dissolved in a 2:1-mixture of saliva/phosphate buffer (pH 7, 4.0 ml) at the indicated concentrations. After 4 h of incubation at 37° C., the aqueous medium is extracted with MTBE (4.0 ml) and the amount of released organoleptic compound determined by quantitative GC-analysis.

TABLE 4
Release of organoleptic alcohol by cleavage in saliva

| | released | conc of | conc. of released | |
|---|---|---|---|---|
| | organoleptic | substrate | organoleptic | % of |
| substrate | compound | [μM] | compound [μM] | theory |
| 4 (Ex. 4) | methyl salicylate | 400 | 169 | 42 |
| 4 (Ex. 4) | methyl salicylate | 200 | 89 | 45 |
| 4 (Ex. 4) | methyl salicylate | 100 | 34 | 34 |
| 6 (Ex. 6) | cinnamic alcohol | 400 | 149 | 37 |
| 6 (Ex. 6) | cinnamic alcohol | 200 | 78 | 39 |

As can be seen from the results given in Table 4, about 40% of the organoleptic alcohol of the theory will be released within 4 hours.

### EXAMPLE 12

### Time Dependent Release of Organoleptic Compound in the Presence of Saliva

A 500 μM solution of 2-ethoxy-4-formylphenyl ethyl fumarate in a 2:1-mixture of saliva/phosphate buffer (pH 7, 4.0 ml) is prepared and incubated at 37° C. Samples of 0.50 ml are withdrawn at the indicated time intervals and extracted with MTBE (0.50 ml). The amount of released ethyl vanillin is determined by quantitative GC-analysis. The results are given below in Table 5.

TABLE 5
Time dependent release

| | conc. of released | |
|---|---|---|
| time [min] | ethy vanillin[μM] | % of theory |
| 30 | 192 | 38 |
| 60 | 235 | 47 |
| 120 | 310 | 62 |

### EXAMPLE 13

### Application Examples

### A) Toothpaste, Opaque

| Ingredients | weight % |
|---|---|
| Glycerol 98% | 3.00 |
| Thickener (Cellulose Gum CMC Blanose 7MFD, | 0.25 |
| Aqualon Company, Hercules, FR) | |
| Sorbitol 70% | 50.00 |
| Sodium Monofluorophosphate | 0.75 |
| Preservatives | 0.20 |
| Sodium Saccharin | 0.10 |
| Silica (Syloblanc 81) (GRACE, Germany) | 6.00 |
| Silica (Syloblanc 82) (GRACE, Germany) | 10.00 |
| Thixotropic Agent (Aerosil 200, Degussa, DE) | 2.00 |
| Titanium Dioxide (Fluka, CH) | 0.60 |
| Sodium Laurylsulfate (Fluka, CH) | 1.50 |
| Mint oil arvensis | 1.00 |
| Compound 1 (Example 1) | 0.6 |
| Purified Water | Ad 100.00 |

### B) Mouthwash

| Ingredients | Weight % |
|---|---|
| Glycerol (87%) | 4.00 |
| Sorbitol (70% solution) | 8.00 |
| Sodium Saccharin | 0.01 |
| Colour (1% solution) | 0.04 |
| Solubilizer Cremophor RH 410 (BASF) | 0.13 |
| Alcohol | 7.00 |
| Mint oil | 0.16 |
| Compound 1 (Example 1) | 0.16 |
| Deionised Water | Ad 100.00 |

### C) Sugarless Chewing Gum

| Ingredients: | Weight % |
|---|---|
| Gum base Valencia-T (Cafosa Gum SA., 08029 | 32.0 |
| Barcelona, Spain) | |
| Sorbitol powder | 47.5 |
| Lycasin concentrated | 8.0 |
| Glycerol | 1.25 |
| Mannitol powder | 4.0 |
| Xylitol milled | 4.0 |
| Aspartame | 0.2 |
| Acesulfame K | 0.05 |
| Mint oil | 2.0 |
| Compound 1 (Example 1) | 1.0 |

1. A compound of formula (I) 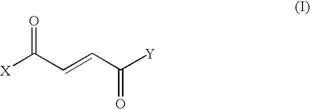
wherein
X is the residue of an organoleptic alcohol comprising 8 to 15 carbon atoms; or
X is the residue of an alcohol, diol, triol or polyol comprising 2 to 7 carbon atoms; and
Y is the residue of an organoleptic alcohol comprising 8 to 15 carbon atoms;
and the compounds of formula (I) having a CLogP of 4.5 or lower.

2. A compound of formula (I) according to claim 1
wherein
X is the residue R′—O of an organoleptic alcohol of the formula R¹—OH, wherein R¹ is selected from the group consisting of
I) saturated and unsaturated, linear and branched, C₈-C₁₅ hydrocarbon residues, optionally containing one or more hydroxyl, carbonyl, carboxyl, and or ether group(s);
II) C₈-C₁₃ hydrocarbon residue containing one ring structure selected from alicyclic C₅, alicyclic C₆, phenol, bicyclic C₇, furan, and spirocyclic C₉ wherein one ring member is an oxygen,
and wherein the C₈-C₁₃ hydrocarbon residue optionally contains one or more hydroxyl, carbonyl, carboxyl, and or ether group(s); or
X is the residue R²—O of ascorbic acid or an alkanol R²—OH, wherein R² is saturated or unsaturated, linear or branched C₂-C₇ alkyl optionally containing one or more hydroxyl, ether, and/or carbonyl group(s), or R² is a C₃-C₇ cycloalkyl optionally containing one or more hydroxyl and/or carbonyl group(s); and
Y is the residue R³—O of an organoleptic alcohol of the formula R³—OH, wherein R³ is selected from the group consisting of
I) saturated and unsaturated, linear and branched, C₈-C₁₅ hydrocarbon residues, optionally containing one or more hydroxyl, carbonyl, carboxyl, and or ether group(s);
II) C₈-C₁₃ hydrocarbon residue containing one ring structure selected from alicyclic C₅, alicyclic C₆, phenol, bicyclic C₇, furan, and spirocyclic C₉ wherein one ring member is an oxygen,
and wherein the C₈-C₁₃ hydrocarbon residue optionally containing one or more hydroxyl, carbonyl, carboxyl, and or ether group(s);
the compounds of formula (I) having a C_{Log}P of 4.5 or lower.

3. A compound according to claim 2 wherein X is the residue R²—O of an alkanol R²—OH selected from the list consisting of ethanol, propanol, propylene glycol, glycerol, sorbitol, xylitol, lactic acid, alpha-glucose and ascorbic acid.

4. A compound according to claim 1 wherein Y is the residue R³—O of an organoleptic alcohol R³—OH selected from:
2-isopropyl-5-methylcyclohexanol,
1,7,7-trimethyl-bicyclo[2.2.1]heptan-2-ol,
4-allyl-2-methoxy-phenol,
2-isopropenyl-5-methylcyclohexan-1-ol,
2-isopropyl-5-methyl-phenol and
6,6-dimethyl-2-methylene-bicyclo[3.1.1]heptan-3-ol.

5. A compound according to claim 1 selected from the group consisting of:
2,3-dihydroxypropyl 2-isopropyl-5-methylcyclohexyl fumarate,
ethyl 2-methyl-4-oxo-4H-pyran-3-yl fumarate,
2-ethoxy-4-formylphenyl ethyl fumarate,
methyl 2-((E)-3-(ethoxycarbonyl)acryloyloxy)-benzoate,
2,3,4,5,6-pentahydroxyhexyl 2-isopropyl-5-methylcyclohexyl fumarate,
cinnamyl ethyl fumarate and
ethyl (Z)-hex-3-enyl fumarate.

6. An oral composition comprising a compound of formula (I) according to claim 1.

7. An oral composition according to claim 6 wherein the oral composition is selected from chewing gum, candies, edible films, beverages and oral care products.

8. (canceled)

9. A method of counteracting oral malodour by providing a compound of formula (I) according to claim 1 to the oral cavity.

10. A method of counteracting oral malodour by providing an oral care product comprising an effective amount of at least one compound of formula (I) according to claim 1, to the oral cavity.

11. A compound according to claim 2 wherein Y is the residue R³—O of an organoleptic alcohol R³—OH selected from:
2-isopropyl-5-methylcyclohexanol,
1,7,7-trimethyl-bicyclo[2.2.1]heptan-2-ol,
4-allyl-2-methoxy-phenol,
2-isopropenyl-5-methylcyclohexan-1-ol,
2-isopropyl-5-methyl-phenol and
6,6-dimethyl-2-methylene-bicyclo[3.1.1]heptan-3-ol.

12. An oral composition comprising a compound of formula (I) according to claim 1.

13. An oral composition according to claim 12 wherein the oral composition is selected from chewing gum, candies, edible films, beverages and oral care products.

14. A method of counteracting oral malodour by providing a compound of formula (I) according to claim 2 to the oral cavity.

15. A method of counteracting oral malodour by providing an oral care product comprising an effective amount of at least one compound of formula (I) according to claim 2 to the oral cavity.
